(19) United States
(12) Patent Application Publication
(10) Pub. No.: US 2004/0248809 A1
(43) Pub. Date: Dec. 9, 2004

(54) Use of antagonists of the bradykinin B2 receptor for the treatment of osteoarthrosis

(75) Inventors: Martin Michaelis, Frankfurt (DE); Karl Rudolphi, Mainz (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/773,772

(22) Filed: Feb. 6, 2004

Related U.S. Application Data
(60) Provisional application No. 60/480,246, filed on Jun. 20, 2003.

(30) Foreign Application Priority Data
Feb. 7, 2003 (DE) 10304994.0-41

(51) Int. Cl. A61K 38/08

(52) U.S. Cl. 514/15

(57) ABSTRACT

Peptides having bradykinin-antagonistic action are suitable for the production of pharmaceuticals for the prophylaxis and therapy of diseases in whose course an increased activity of matrix metalloproteinases is involved. These include diseases such as degenerative joint diseases, for example osteoarthrosis, spondylosis and chondroporosis after joint trauma or relatively long immobilization of a joint after meniscus or patella injuries or torn ligaments. The invention therefore relates to the use of a compound of the formula I,

A-B-X-E-F-K-(D)-TIC-G-M-F′-I (I)

for the production of pharmaceuticals for the treatment of degenerative joint diseases, wherein A, B, X, E, F, K, (D)-TIC, G, M, F′ and I are as defined herein.

This application claims the benefit of U.S. Provisional Application No. 60/480,246, filed Jun. 20, 2003.

### FIELD OF THE INVENTION

The invention relates to the use of peptides having bradykinin-antagonistic action for the production of pharmaceuticals for the treatment of degenerative joint diseases.

### BACKGROUND OF THE INVENTION

In degenerative joint diseases such as osteoarthrosis, a slowly progressing destruction of the joint takes place, which is caused in particular by the proteolytic degradation of collagen by collagenases. Collagenases belong to the superfamily of the metalloproteinases (MP) or matrix metalloproteinases (MMPs). MMPs are capable of degrading fibrillar and nonfibrillar collagen and proteoglycans, which are all important constituents of the cartilaginous matrix. MMP 3 is involved in the biological degradation of the extracellular matrix and is found in increased levels in patients with osteoarthrosis, which is why particular importance is ascribed to MMP 3 in the degradation of the joint matrix in osteoarthrosis (Manicourt et al. (1994) Arthritis and Rheumatism 37:1774-83).

Bradykinin is a naturally occurring nonapeptide that has some pharmacological effects that lead to inflammation and pain. Peptides having bradykinin-antagonistic action have already been described in European patent EP 0 370 453 B1. It is further known that peptides having bradykinin-antagonistic action can be employed in the treatment of osteoarthritis or rheumatoid arthritis (AU 638 350). Osteoarthritis and rheumatoid arthritis are joint diseases having severe inflammatory phases in the course of the disease. Lerner et al. (Arthritis and Rheumatism (1987), 30, 530-540) report that, in the context of rheumatoid arthritis, bradykinin may actually enhance bone resorption, but does not stimulate the degradation of the cartilaginous matrix itself.

In the attempt to find active compounds for the treatment of degenerative joint diseases, it has now been found that the peptide employed according to the invention inhibits the release of MMPs such as MMP-3 (and MMP-1 and MMP-13). As a result, the matrix degradation can be inhibited significantly more effectively than only by the inhibition of MMPs themselves that have already been released or formed in the tissue.

### SUMMARY OF THE INVENTION

The invention therefore relates to the use of the compound of the formula I,

A-B-X-E-F-K-(D)-TIC-G-M-F′-I (I)

for the production of pharmaceuticals for the treatment of degenerative joint diseases, in which:

A a₁) is a hydrogen atom, (C₁-C₈)-alkyl, (C₁-C₈)-alkanoyl, (C₁-C₈)-alkoxycarbonyl or (C₁-C₈)-alkylsulfonyl, in which in each case 1, 2 or 3 hydrogen atoms are optionally replaced by 1, 2 or three identical or different radicals from the group consisting of carboxyl, amino, (C₁-C₄)-alkyl, (C₁-C₄)-alkylamino, hydroxy, (C₁-C₃)-alkoxy, halogen, di-(C₁-C₄)-alkylamino, carbamoyl, sulfamoyl, (C₁-C₄)-alkoxycarbonyl, (C₆-C₁₂)-aryl and (C₆-C₁₂)-aryl-(C₁-C₅)-alkyl,

or in which in each case 1 hydrogen atom is optionally replaced by a radical from the group consisting of (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl and (C₃-C₉)-heteroaryloxy and 1 or 2 hydrogen atoms are replaced by 1 or 2 identical or different radicals from the group consisting of carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl and (C₆-C₁₂)-aryl-(C₁-C₅)-alkyl,

a₂) is (C₃-C₈)-cycloalkyl, carbamoyl,

which can optionally be substituted on the nitrogen by (C₁-C₆)-alkyl or (C₆-C₁₂)-aryl,

(C₆-C₁₂)-aryl, (C₆-C₁₂)-aroyl, (C₆-C₁₂)-arylsulfonyl or (C₃-C₉)-heteroaryl or (C₃-C₉)heteroaroyl, where in the radicals defined under a₁) and a₂) heteroaryl, aroyl, arylsulfonyl and heteroaroyl in each case is optionally substituted by 1, 2, 3 or 4 different radicals from the group consisting of carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl and (C₁-C₄)-alkoxycarbonyl, or

a₃) is a radical of the formula II,

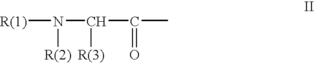

where

R(1) is defined as A under a₁) or a₂),

R(2) is a hydrogen atom or methyl,

R(3) is a hydrogen atom or (C₁-C₆)-alkyl, where alkyl is unsubstituted or monosubstituted by amino, substituted amino, hydroxy, carbamoyl, guanidino, substituted guanidino, ureido, mercapto, methylmercapto, phenyl, 4-chlorophenyl, 4-fluorophenyl, 4-nitrophenyl, 4-methoxyphenyl, 4-hydroxyphenyl, phthalimido, 4-imidazolyl, 3-indolyl, 2-thienyl, 3-thienyl, 2-pyridyl, 3-pyridyl or cyclohexyl,

where substituted amino is a moiety —NH-A′- and substituted guanidino is a moiety —NH—C(NH)—NH-A′-, in which A′ is as defined under a₁) or a₂);

B is Arg, Lys, Orn, 2,4-diaminobutyroyl or an L-homoarginine radical,

where in each case the amino or the guanidino group of the side chain can be substituted by an A as described under a₁) or a₂);

X is a compound of the formula IIIa or IIIb

G′-G′-Gly (IIIa)

G′-NH—(CH₂)ₙ—CO (IIIb),

in which G′ independently of one another is a radical of the formula IV

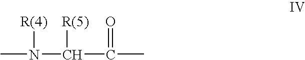

in which R(4) and R(5) together with the atoms carrying these is a heterocyclic mono-, bi- or tricyclic ring system having 2 to 15 carbon atoms, and n is 2 to 8;

E is the radical of phenylalanine,

which is optionally substituted by halogen in the 2-, 3- or 4-ring position, or

is tyrosine, O-methyltyrosine, 2-thienylalanine, 2-pyridylalanine or naphthylalanine;

F independently of one another is the radical of a neutral, acidic or basic aliphatic or aromatic amino acid,

which can be substituted in the side chain,

or is a covalent bond;

(D)-Tic is the radical of the formula V

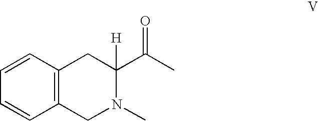

G is G′ or a covalent bond;

F′ is the radical of a basic amino acid Arg or Lys in the L or D form or a covalent bond,

where the guanidino group or amino group of the side chain can be substituted by A as defined under a₁) or a₂),

or is a radical —NH—(CH₂)ₙ— where n is 2-8,

or a covalent bond;

I is —OH, —NH₂ or NHC₂H₅;

K is the radical —NH—(CH₂), —CO where x is 1 to 4 or a covalent bond;

M is defined as F,

and its physiologically tolerable salts.

### DETAILED DESCRIPTION OF THE INVENTION

Definition of Terms

As used above, and throughout the description of the invention, the following terms, unless otherwise indicated, shall be understood to have the following meanings.

The term “(C₁-C₈)-alkyl” is understood as meaning hydrocarbon radicals whose carbon chain is straight-chain or branched and contains 1 to 8 carbon atoms, for example methyl, ethyl, propyl, isopropyl, butyl, isobutyl, tertiary-butyl, pentyl, isopentyl, neopentyl, hexyl, 2,3-dimethylbutyl, heptyl, neohexyl or octyl.

The term “halogen” is understood as meaning fluorine, chlorine, bromine or iodine.

The term “(C₃-C₈)-cycloalkyl” is understood as meaning radicals such as moieties that are derived from 3- to 8-membered monocycles such as cyclopropyl, cyclobutyl, cyclopentyl, cyclohexyl, cycloheptyl or cyclooctyl.

The term “—(C₆-C₁₂)-aryl” is understood as meaning aromatic hydrocarbon radicals having 6 to 14 carbon atoms in the ring. —(C₆-C₁₂)-aryl radicals are, for example, phenyl, naphthyl, for example 1-naphthyl, 2-naphthyl, biphenylyl, for example 2-biphenylyl, 3-biphenylyl and 4-biphenylyl, anthryl or fluorenyl. Biphenylyl radicals, naphthyl radicals and in particular phenyl radicals are preferred aryl radicals.

The term “(C₃-C₉)-heteroaryl” is to be understood as meaning radicals such as acridinyl, azetidinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuran[2,3-b]-tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pyridooxazolyl, pyridoimidazolyl, pyridothiazolyl, pyridothiophenyl, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetra-hydrofuranyl, tetrahydroisoquinolinyl, tetrahydroquinolinyl, 6H-1,2,5-thia-diazinyl, 1,2,3-thiadiazolyl, 1,2,4-thiadiazolyl, 1,2,5-thiadiazolyl, 1,3,4-thia-diazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. The preferred examples are pyridyl; such as 2-pyridyl, 3-pyridyl or 4-pyridyl; pyrrolyl; such as 2-pyrrolyl and 3-pyrrolyl; furyl; such as 2-furyl and 3-furyl; thiophenyl, thienyl; such as 2-thienyl and 3-thienyl; imidazolyl, pyrazolyl, oxazolyl, isoxazolyl, thiazolyl, isothiazolyl, tetrazolyl, pyridazinyl, pyrazinyl, pyrimidinyl, indolyl, isoindolyl, benzofuranyl, benzothiophenyl, 1,3-benzodioxolyl, indazolyl, benzimid-azolyl, benzoxazolyl, benzothiazolyl, quinolinyl, isoquinolinyl, chromanyl, isochromanyl, cinolinyl quinazolinyl, quinoxalinyl, phthalazinyl, pyrido-imidazolyl, pyridopyridinyl, pyridopyrimidinyl, purinyl and pteridinyl.

Patient includes both human and other mammals.

Pharmaceutically effective amount means an amount of the compound according to the invention effective in producing the desired therapeutic effect.

Preferred or Particular Embodiment

A particular embodiment of the invention is the use according to the invention of the compound of the formula I, wherein:

B is Arg, Orn or Lys,

where the guanidino group or the amino group of the side chain is unsubstituted or can be substituted by (C₁-C₈)-alkanoyl, (C₆-C₁₂)-aroyl, (C₃-C₉)-heteroaroyl, (C₁-C₈)-alkylsulfonyl or (C₆-C₁₂)-arylsulfonyl, where the aryl, heteroaryl, aroyl, arylsulfonyl and hetero-aroyl radicals can be substituted as described above under a₂) by optionally 1, 2, 3 or 4 identical or different radicals;

E is phenylalanine, 2-chlorophenylalanine, 3-chlorophenyl-alanine, 2-fluorophenylalanine, 3-fluorophenylalanine, 4-fluorophenylalanine, tyrosine, O-methyltyrosine or β-(2-thienyl)alanine;

K is a covalent bond; and

M is a covalent bond.

A more particular embodiment of the invention is the use according to the invention of the compound of the formula I, wherein:

A is a hydrogen atom, (D)- or (L)-H-Arg, (D)- or (L)-H-Lys or (D)- or (L)—H-Orn;

B is Arg, Orn or Lys,

where the guanidino group or the amino group of the side chain can be substituted by a hydrogen atom, (C₁-C₈)-alkanoyl, (C₆-C₁₂)-aroyl, (C₃-C₉)-heteroaroyl, (C₁-C₈)-alkylsulfonyl or (C₆-C₁₂)-arylsulfonyl, where the aryl, heteroaryl, aroyl, arylsulfonyl and heteroaroyl radicals can optionally, be substituted by 1, 2, 3 or 4 identical or different radicals from the group consisting of methyl, methoxy and halogen;

X is Pro-Pro-Gly, Hyp-Pro-Gly or Pro-Hyp-Gly;

E is Phe or Thia;

F is Ser, Hser, Lys, Leu, Val, Nle, Ile or Thr;

K is a covalent bond

M is a covalent bond

G is the radical of a heterocyclic ring system of the formula IV, selected from the radicals of the heterocycles pyrrolidine (A), piperidine (B), tetrahydroisoquinoline (C), cis- or trans-decahydroisoquinoline (D), cis-endo-octahydroindole (E), cis-exo-octahydroindole (E), trans-octahydroindole (E), cis-endo-, cis-exo-, trans-octahydrocyclopentano[b]pyrrole (F), or hydroxyproline (V);

F′ is Arg; and

I is OH.

A further particular embodiment of the invention is the use according to the invention of a compound of the formula I, which is selected from the group:

H-(D)-Arg-Arg-Pro-Hyp-Gly-Thia-Ser-(D)-Tic-Oic-Arg-OH,

H-(D)-Arg-Arg-Pro-Pro-Gly-Thia-Ser-(D)-Tic-Oic-Arg-OH,

H-(D)-Arg-Arg-Pro-Hyp-Gly-Phe-Ser-(D)-Tic-Oic-Arg-OH,

H-(D)-Arg-Arg-Hyp-Pro-Gly-Phe-Ser-(D)-Tic-Oic-Arg-OH and

H-(D)-Arg-Arg-Pro-Pro-Gly-Phe-Ser-(D)-Tic-Oic-Arg-OH.

A further particular embodiment of the invention is the use according to the invention of a compound of the formula I, which the compound of the formula I is

D-arginyl-L-arginyl-L-prolyl-L-prolylglycyl-3-(2-thienyl)-L-alanyl-L-seryl-(3R)-1,2,3,4-tetrahydro-3-isoquinolinecarbonyl-(2S,3aS,7aS)-octahydro-1H-indole-2-carbonyl-L-arginine, also known under the name HOE 140.

The peptides employed according to the invention are prepared as described in EP 0 370 453 B1.

On account of the pharmacological properties, the compounds according to the invention are suitable for the selective prophylaxis and therapy of degenerative joint diseases such as osteoarthrosis; spondylosis or cartilage atrophy after immobilization such as after joint trauma or relatively long immobilization of a joint after meniscus or patella injuries or torn ligaments. The term “osteoarthrosis” is understood as meaning a disease which chiefly develops in connection with a disparity between the strain on and the load capacity of the individual joint components and joint tissues, which is associated with increasing destruction of the cartilage and which is in the main not inflammatory. Damage to the joint cartilage, such as fraying, demedullation and hyalinization, followed by reactive changes in the subchondral bone, and also capsule changes, is prominent in the pathology. The term “spondylosis” is understood as meaning an arthrosis of the vertebral bodies, with this arthrosis being characterized by a noninflammatory loss of cartilage from the vertebral bodies and intervertebral disks.

The pharmaceuticals according to the invention can be administered by inhalative or transdermal administration or by subcutaneous, intraarticular, intraperitoneal or intravenous injection. Intraarticular administration or topical application is preferred.

Suitable solid or pharmaceutical preparation forms are, for example, suspensions, emulsions, or injectable solutions, and preparations having protracted release of active compound, in whose preparation customary excipients are used.

Preferably, the pharmaceutical preparations are prepared and administered in dose units, each unit containing as active constituent a certain dose of the compound of the formula I according to the invention. In the case of injection solutions in ampoule form, this dose can be up to approximately 300 mg, but preferably approximately 10 to 100 mg, in the case of injection solutions for intraarticular treatment up to approximately 300 micrograms, preferably 100 micrograms.

For the treatment of an adult patient, depending on the activity of the compound according to formula I, daily doses of approximately 0.01 mg/kg to 10 mg/kg of active compound are indicated in the case of systemic administration, in the case of the administration of injection solutions daily doses of 0.001 mg/kg to 0.005 mg/kg of active compound are indicated and in the case of topical or inhalative administration, daily doses of 0.01 mg/kg to 5 mg/kg of active compound are indicated. Under certain circumstances however, higher or lower daily doses may also be appropriate. The daily, dose can be administered either by single administration in the form of an individual dose unit or else a number of smaller dose units or by multiple administration of subdivided doses at specific intervals.

### EXAMPLES

The invention is illustrated below with the aid of examples.

The abbreviations used for the amino acids correspond to the three-letter code customary in peptide chemistry, as is described in Europ. 4. Biochem. 138, 9 (1984). Further abbreviations used are listed below.

| | |
|---|---|
| Oic | octahydro-1H-indole-2-carbonyl |
| Thia | 2-thienylalanyl |
| Tic | 1,2,3,4-tetrahydroisoquinolin-3-ylcarbonyl |

HOE 140 was prepared as described in EP 0 370 453 B1.

### PHARMACOLOGICAL EXAMPLES

For the analysis of the disease-modifying action of HOE140 in a cell culture model relevant to cartilage, the MMP3 expression was analyzed in the chondrosarcoma cell line SW1353 (ATCC: HTB 94). For the experiments, SW1353 cells were cultured under standard conditions (37° C., 5% CO₂) in DMEM-Glutamax with 10% of fetal calf serum (FCS) in plastic culture bottles. After detrypsinization of the cells, 50,000 cells were inoculated per well of a 96-well flat-bottom plate in medium without FCS and preincubated with the compound HOE140 in an incubator. After one hour, the cells were stimulated by addition of human IL1-β (0.1 ng/mL, Roche) in a total volume of 300 μL. After incubation for 24 hours under standard conditions, the cell culture supernatant was taken off, centrifuged for 5 minutes and frozen at −20° C. until further analysis. The MMP3 expression in the cell culture supernatants was then analyzed by means of a commercial MMP3 ELISA test system (Amersham) according to the instructions of the manufacturer. In parallel to this, a WST cytotoxicity test was carried out with the remaining cells. For this the commercial test system of Roche was used and the measurement was carried out according to the instructions of the >manufacturer's protocol. Table 1 below shows the results. Bradykinin increases the MMP3 release by more than 30%. This increased release of MMP3 was inhibited by HOE140 in a dose-dependent manner.

TABLE 1
MMP-3 release from SW cells

| | MMP-3 release | | Relative values |
|---|---|---|---|
| Stimulation | MW | | based on |
| parameter | (OD 450 nm) | SD | starting value |
| unstimulated | 93 | 20 | |
| IL1α (0.05 ng/mL) | 328 | 17 | 100 |
| IL1α + bradykinin | 433 | 32 | 132.0 |
| (0.1 μM) | | | |
| IL1α + bradykinin | 458 | 50 | 139.6 |
| (0.1 μM) + 0.05 μM HOE140 | | | |
| IL1α + bradykinin | 371 | 8 | 113.1 |
| (0.1 μM) + 0.1 μM HOE140 | | | |
| IL1α + bradykinin | 309 | 18 | 94.2 |
| (0.1 μM) + 0.5 μM HOE140 | | | |
| IL1α + bradykinin | 306 | 27 | 933 |
| (0.1 μM) + 1 μM HOE140 | | | |

We claim:

1. A method for treating a degenerative joint disease, in a patient in need thereof, comprising administering to the patient a pharmaceutically effective amount of a compound of formula I
A-B-X-E-F-K-(D)-TIC-G-M-F′-I (I)
wherein:
A is hydrogen, (C₁-C₈)-alkyl, (C₁-C₈)-alkanoyl, (C₁-C₈)-alkoxycarbonyl or (C₁-C₈)-alkylsulfonyl, each of which is optionally substituted one, two or three times by carboxyl, amino, (C₁-C₄)-alkyl, (C₁-C₄)-alkyl-amino, hydroxy, (C₁-C₃)-alkoxy, halogen, di-(C₁-C₄)-alkyl-amino, carbamoyl, sulfamoyl, (C₁-C₄)-alkoxycarbonyl, (C₆-C₁₂)-aryl or (C₆-C₁₂)-aryl-(C₁-C₅)-alkyl, or each of which is optionally substituted one time by (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl or (C₃-C₉)-heteroaryloxy, and is further optionally substituted one or two times by carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl or (C₆-C₁₂)-aryl-(C₁-C₅)-alkyl, wherein the heteroaryl is optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl,
(C₃-C₈)-cycloalkyl,
carbamoyl, which is optionally substituted on the nitrogen by (C₁-C₆)-alkyl or (C₆-C₁₂)-aryl,
(C₆-C₁₂)-aryl, (C₆-C₁₂)-aroyl, (C₆-C₁₂)-arylsulfonyl, (C₃-C₉)-heteroaryl or (C₃-C₉)heteroaroyl, wherein the heteroaryl, aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl, or
of formula II, 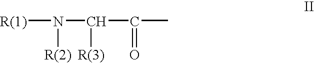
wherein
R(1) is hydrogen,
(C₁-C₈)-alkyl, (C₁-C₈)-alkanoyl, (C₁-C₈)-alkoxycarbonyl or (C₁-C₈)-alkylsulfonyl, each of which is optionally substituted one, two or three times by carboxyl, amino, (C₁-C₄)-alkyl, (C₁-C₄)-alkyl-amino, hydroxy, (C₁-C₃)-alkoxy, halogen, di-(C₁-C₄)-alkyl-amino, carbamoyl, sulfamoyl, (C₁-C₄)-alkoxycarbonyl, (C₆-C₁₂)-aryl or (C₆-C₁₂)-aryl-(C₁-C₅)-alkyl, or each of which is optionally substituted one time by (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl or (C₃-C₉)-heteroaryloxy, and is further optionally substituted one or two times by carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl or (C₆-C₁₂)-aryl-(C₁-C₅)-alkyl, wherein the heteroaryl is optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl,
(C₃-C₈)-cycloalkyl, carbamoyl, which is optionally substituted on the nitrogen by (C₁-C₆)-alkyl or (C₆-C₁₂)-aryl, or
(C₆-C₁₂)-aryl, (C₆-C₁₂)-aroyl, (C₆-C₁₂)-arylsulfonyl, (C₃-C₉)-heteroaryl or (C₃-C₉)heteroaroyl, wherein the heteroaryl, aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl,
R(2) is hydrogen or methyl,
R(3) is hydrogen or (C₁-C₆)-alkyl, wherein the alkyl is optionally monosubstituted by amino, substituted amino, hydroxy, carbamoyl, guanidino, substituted guanidino, ureido, mercapto, methyl-mercapto, phenyl, 4-chlorophenyl, 4-fluorophenyl, 4-nitrophenyl, 4-methoxyphenyl, 4-hydroxyphenyl, phthalimido, 4-imidazolyl, 3-indolyl, 2-thienyl, 3-thienyl, 2-pyridyl, 3-pyridyl or cyclohexyl, wherein the substituted amino is —NH-A′- and the substituted guanidino is —NH—C(NH)—NH-A′-, wherein A′ is
hydrogen,
(C₁-C₈)-alkyl, (C₁-C₈)-alkanoyl, (C₁-C₈)-alkoxycarbonyl or (C₁-C₈)-alkylsulfonyl, each of which is optionally substituted one, two or three times by carboxyl, amino, (C₁-C₄)-alkyl, (C₁-C₄)-alkyl-amino, hydroxy, (C₁-C₃)-alkoxy, halogen, di-(C₁-C₄)-alkyl-amino, carbamoyl, sulfamoyl, (C₁-C₄)-alkoxycarbonyl, (C₆-C₁₂)-aryl or (C₆-C₁₂)-aryl-(C₁-C₅)-alkyl, or each of which is optionally substituted one time by (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl or (C₃-C₉)-heteroaryloxy, and is further optionally substituted one or two times by carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl or (C₆-C₁₂)-aryl-(C₁-C₅)-alkyl, wherein the heteroaryl is optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl,
(C₃-C₈)-cycloalkyl,
carbamoyl, which is optionally substituted on the nitrogen by (C₁-C₆)-alkyl or (C₆-C₁₂)-aryl, or
(C₆-C₁₂)-aryl, (C₆-C₁₂)-aroyl, (C₆-C₁₂)-arylsulfonyl, (C₃-C₉)-heteroaryl or (C₃-C₉)heteroaroyl, wherein the heteroaryl, aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl;
B is Arg, Lys, Orn, 2,4-diaminobutyroyl or L-homo-arginine,
wherein the amino or the guanidino group of the side chain of Arg, Lys, Orn, 2,4-diaminobutyroyl or L-homo-arginine is independently optionally substituted by
hydrogen,
(C₁-C₈)-alkyl, (C₁-C₈)-alkanoyl, (C₁-C₈)-alkoxycarbonyl or (C₁-C₈)-alkylsulfonyl, each of which is optionally substituted one, two or three times by carboxyl, amino, (C₁-C₄)-alkyl, (C₁-C₄)-alkyl-amino, hydroxy, (C₁-C₃)-alkoxy, halogen, di-(C₁-C₄)-alkyl-amino, carbamoyl, sulfamoyl, (C₁-C₄)-alkoxycarbonyl, (C₆-C₁₂)-aryl or (C₆-C₁₂)-aryl-(C₁-C₅)-alkyl, or each of which is optionally substituted one time by (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl or (C₃-C₉)-heteroaryloxy, and is further optionally substituted one or two times by carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl or (C₆-C₁₂)-aryl-(C₁-C₅)-alkyl, wherein the heteroaryl is optionally substituted one, two, three or four times by carboxyl, amino, nitro, hydroxy, cyano, (C₁-C₄)-alkylamino, (C₁-C₄)-alkyl, (C₉-C₄)-alkoxy, halogen, di-(C₁-C₄)-alkylamino, carbamoyl, sulfamoyl or (C₁-C₄)-alkoxycarbonyl,
(C₃-C₈)-cycloalkyl,
carbamoyl, which is optionally substituted on the nitrogen by (C₁-C₆)-alkyl or (C₆-C₁₂)-aryl, or
(C₆-C₁₂)-aryl, (C₆-C₁₂)-aroyl, (C₆-C₁₂)-arylsulfonyl, (C₃-C₉)-heteroaryl or (C₃-C₉)heteroaroyl, wherein the heteroaryl, aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl;
X is of formula IIIa or IIIb
G′-G′-Gly (IIIa)
G′-NH—(CH₂)ₙ—CO (IIIb),
wherein G′ independently of one another is of formula IV 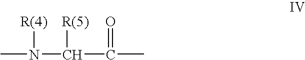
wherein R(4) and R(5) together with the atoms they connect to form a heterocyclic mono-, bi- or tricyclic ring having 2 to 15 carbon atoms, and n is 2 to 8;
E is phenylalanine optionally substituted by halogen in the 2-, 3-or 4-ring position, tyrosine, O-methyltyrosine, 2-thienylalanine, 2-pyridylalanine or naphthylalanine;
F is covalent bond, or neutral, acidic or basic aliphatic or aromatic amino acid, which is optionally substituted in the side chain;
(D)-TIC is of formula V 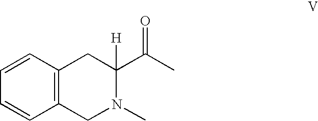
G is G′ or a covalent bond;
F′ is covalent bond, —NH—(CH₂)ₙ— wherein n is 2-8, or basic amino acid Arg or Lys in the L or D form, wherein the guanidino group or amino group of the side chain of the Arg or Lys is optionally substituted by
hydrogen,
(C₁-C₈)-alkyl, (C₁-C₈)-alkanoyl, (C₁-C₈)-alkoxycarbonyl or (C₁-C₈)-alkylsulfonyl, each of which is optionally substituted one, two or three times by carboxyl, amino, (C₁-C₄)-alkyl, (C₁-C₄)-alkyl-amino, hydroxy, (C₁-C₃)-alkoxy, halogen, di-(C₁-C₄)-alkyl-amino, carbamoyl, sulfamoyl, (C₁-C₄)-alkoxycarbonyl, (C₆-C₁₂)-aryl or (C₆-C₁₂)-aryl-(C₁-C₅)-alkyl, or each of which is optionally substituted one time by (C₃-C₈)-cycloalkyl, (C₁-C₄)-alkylsulfonyl, (C₁-C₄)-alkylsulfinyl, (C₆-C₁₂)-aryl-(C₁-C₄)-alkylsulfonyl, (C₆-C₁₂)-aryl-(C₁-C₄)-alkylsulfinyl, (C₆-C₁₂)-aryloxy, (C₃-C₉)-heteroaryl or (C₃-C₉)-heteroaryloxy, and is further optionally substituted one or two times by carboxyl, amino, (C₁-C₄)-alkylamino, hydroxy, (C₁-C₄)-alkoxy, halogen, di-(C₁-C₄)-alkylamino, carbamoyl, sulfamoyl, (C₁-C₄)-alkyloxycarbonyl, (C₆-C₁₂)-aryl or (C₆-C₁₂)-aryl-(C₁-C₅)-alkyl, wherein the heteroaryl is optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl,
(C₃-C₈)-cycloalkyl,
carbamoyl, which is optionally substituted on the nitrogen by (C₁-C₆)-alkyl or (C₆-C₁₂)-aryl, or
(C₆-C₁₂)-aryl, (C₆-C₁₂)-aroyl, (C₆-C₁₂)-arylsulfonyl, (C₃-C₉)-heteroaryl or (C₃-C₉)heteroaroyl, wherein the heteroaryl, aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl;
I is —OH, —NH₂ or NHC₂H₅;
K is covalent bond or —NH—(CH₂)ₓ—CO, wherein x is 1 to 4; and
M is covalent bon, or neutral, acidic or basic aliphatic or aromatic amino acid, which is optionally substituted in the side chain;
or its physiologically tolerable salts thereof.

2. The method according to claim 1, wherein
B is Arg, Orn or Lys,
wherein the guanidino group or the amino group of the side chain is each independently optionally substituted by (C₁-C₈)-alkanoyl, (C₆-C₁₂)-aroyl, (C₃-C₉)-heteroaroyl, (C₁-C₈)-alkylsulfonyl or (C₆-C₁₂)-arylsulfonyl, wherein the aroyl, arylsulfonyl and heteroaroyl are each independently optionally substituted one, two, three or four times by carboxyl, amino, nitro, hydroxy, cyano, (C₁-C₄)-alkylamino, (C₁-C₄)-alkyl, (C₁-C₄)-alkoxy, halogen, di-(C₁-C₄)-alkylamino, carbamoyl, sulfamoyl or (C₁-C₄)-alkoxycarbonyl;
E is phenylalanine, 2-chlorophenylalanine, 3-chlorophenylalanine, 2-fluorophenylalanine, 3-fluorophenyl-alanine, 4-fluorophenylalanine, tyrosine, O-methyl-tyrosine or β-(2-thienyl)alanine;
K is covalent bond; and
M is covalent bond.

3. The method according to claim 1, wherein:
A is hydrogen, (D)- or (L)-H-Arg, (D)- or (L)-H-Lys or (D)- or (L)-H-Orn;
B is Arg, Orn or Lys,
wherein the guanidino group or the amino group of the side chain is optionally substituted by hydrogen, (C₁-C₈)-alkanoyl, (C₆-C₁₂)-aroyl, (C₃-C₉)-heteroaroyl, (C₁-C₈)-alkylsulfonyl or (C₆-C₁₂)-arylsulfonyl, wherein the aroyl, arylsulfonyl and heteroaroyl are each independently optionally substituted one, two, three or four times by methyl, methoxy or halogen;
X is Pro-Pro-Gly, Hyp-Pro-Gly or Pro-Hyp-Gly;
E is Phe or Thia;
F is Ser, Hser, Lys, Leu, Val, Nle, Ile or Thr;
K is covalent bond
M is covalent bond
G is of the formula IV, 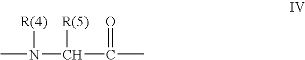
wherein R(4) and R(5) together with the atoms they connect to form pyrrolidine, piperidine, tetrahydro-isoquinoline, cis- or trans-decahydroisoquinoline, cis-endo-octahydroindole, cis-exo-octahydro-indole, trans-octahydroindole, cis-endo-, cis-exo-, trans-octahydrocyclopentano[b]pyrrole, or hydroxyproline;
F′ is Arg; and
I is OH.

4. The method according to claim 1, wherein the compound of the formula I is
H-(D)-Arg-Arg-Pro-Hyp-Gly-Thia-Ser-(D)-Tic-Oic-Arg-OH,
H-(D)-Arg-Arg-Pro-Pro-Gly-Thia-Ser-(D)-Tic-Oic-Arg-OH,
H-(D)-Arg-Arg-Pro-Hyp-Gly-Phe-Ser-(D)-Tic-Oic-Arg-OH,
H-(D)-Arg-Arg-Hyp-Pro-Gly-Phe-Ser-(D)-Tic-Oic-Arg-OH or
H-(D)-Arg-Arg-Pro-Pro-Gly-Phe-Ser-(D)-Tic-Oic-Arg-OH.

5. The method according to claim 1, wherein the compound of the formula I is D-arginyl-L-arginyl-L-prolyl-L-prolylglycyl-3-(2-thienyl)-L-alanyl-L-seryl-(3R)-1,2,3,4-tetrahydro-3-isoquinolinecarbonyl-(2S,3aS,7aS)-octahydro-1H-indole-2-carbonyl-L-arginine.

6. The method according to claim 1, wherein the degenerative joint disease is osteoarthrosis, spondyloses or cartilage atrophy after immobilization.

7. The method according to claim 1, wherein the administration is carried out by subcutaneous, intraarticular, intraperitoneal or intravenous injection or transdermal administration.
